(19) United States
(12) Patent Application Publication
(10) Pub. No.: US 2024/0358695 A1
(43) Pub. Date: Oct. 31, 2024

(54) Pharmaceutical Compositions of Rimegepant

(71) Applicant: Aurobindo Pharma Ltd, Hyderabad (IN)

(72) Inventors: Sreekanth Manikonda, Hyderabad (IN); Avinash Reddy Manda, Hyderabad (IN); Siva Kumar Reddy Chamala, Hyderabad (IN); Nagaprasad Vishnubhotla, Hyderabad (IN); Sivakumaran Meenakshisunderam, Hyderabad (IN)

(73) Assignee: Aurobindo Pharma Ltd, Hyderabad (IN)

(21) Appl. No.: 18/643,177

(22) Filed: Apr. 23, 2024

(30) Foreign Application Priority Data
Apr. 25, 2023 (IN) 202341029746

(51) Int. Cl. A61K 31/4545; A61K 9/20

(52) U.S. Cl. CPC A61K 31/4545; A61K 9/2009; A61K 9/2018; A61K 9/2027; A61K 9/2054; A61K 9/2059; A61K 9/2095

(57) ABSTRACT

The present invention relates to rapidly disintegrating pharmaceutical composition comprising Rimegepant or pharmaceutically acceptable salt thereof, and processes for manufacture thereof.

### CROSS REFERENCE TO RELATED APPLICATION

This application claims priority from an Indian Patent Application IN 202341029746 filed on Apr. 25, 2023.

### FIELD OF THE INVENTION

The present invention relates to rapidly disintegrating pharmaceutical composition comprising Rimegepant or pharmaceutically acceptable salt thereof, and processes for manufacture thereof.

### BACKGROUND OF THE INVENTION

Rimegepant sulfate is described chemically as (5S,6S,9R)-5-amino-6-(2,3-difluorophenyl)-6,7,8,9-tetrahydro-5H-cyclohepta[b]pyridin-9-yl4-(2-oxo-2,3-dihydro-1Himidazo[4,5-b]pyridin-1-yl)-1-piperidinecarboxylate hemisulfate sesquihydrate. Its molecular formula is C₂₈H₂₈F₂N₆O₃ 0.5 H₂SO₄ 1.5 H₂O and its molecular weight is 610.63 and is represented by the following formula.

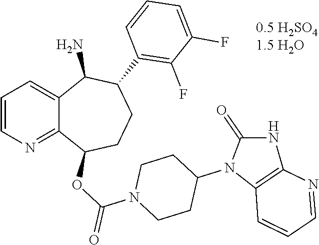 [SMILES: N[C@@H]1c2cccnc2[C@H](OC(=O)N2CCC(n3c(=O)[nH]c4ncccc43)CC2)CC[C@H]1c1cccc(F)c1F]

Rimegepant is commercially available under the brand name NURTEC® ODT in 75 mg orally disintegrating tablets and marketed by Pfizer Inc. in the United States for the treatment of acute treatment of migraine with or without aura in adults and preventive treatment of episodic migraine in adults. NURTEC ODT has been developed using Zydis® orally disintegrating tablet (ODT) technology to create a freeze-dried tablet that disperses almost instantly in the mouth without water.

U.S. Pat. No. 8,314,117 discloses Rimegepant compound generically and specifically; U.S. Pat. No. 8,759,372 discloses Rimegepant Hemisulfate salt and its crystalline form H1.5-1.

U.S. Pat. No. 11,083,724 discloses pharmaceutical composition of Rimegepant and pharmaceutically acceptable salt thereof, wherein the pharmaceutical composition is in a form of an oral solid molded fast-dispersing dosage form. The patent utilizes Zydis® technology to develop oral solid dosage form of Rimegepant that disperses almost instantly in mouth.

There are disadvantages in developing ODT using Zydis® technology in terms of production, storage and transport. Moreover this dosage forms are more costly to produce, e.g., freeze-drying processes are expensive and time-consuming. In addition, the effectiveness of a freeze-drying process depends on the physico-chemical parameters of the active substances used. Finally, they cannot be packaged in a conventional, multi tablet bottle, individual foil pouches or traditional blister packaging due to high likelihood of tablet breakage. This can seriously interfere with the processing efficiencies of high volume presses and ultimately add up to cost for production.

There is no other technology reported in the prior art for developing ODT for Rimegepant which provides rapid action. Therefore there exists a need in the art for alternatives to expensive manufacturing processes, e.g., freeze drying, to produce orally disintegrating Rimegepant tablets, which are bioequivalent to the existing NURTEC® ODT orally disintegrating tablets.

One of the major side effects reported by NURTEC ODT is nausea which affects patient's compliance. Therefore, another motive of the present invention is to provide rapidly disintegrating pharmaceutical composition comprising Rimegepant wherein the dosage form disintegrates in saliva within 30 seconds without experiencing any aftertaste.

### SUMMARY OF THE INVENTION

The present invention provides a rapidly disintegrating tablets composition comprising Rimegepant or a pharmaceutically acceptable salt thereof and one or more pharmaceutically acceptable excipients, prepared by conventional tableting process.

In one aspect, the present invention provides a rapidly disintegrating tablets composition comprising Rimegepant or a pharmaceutically acceptable salt thereof comprising:
(a) an intragranular portion comprising therapeutically effective amount of Rimegepant or a pharmaceutically acceptable salt thereof and at least one diluent, at least one disintegrant and one or more excipients and
(b) an extragranular portion comprising at least one disintegrant; wherein said tablet is not freeze-dried product.

In another aspect, the present invention provides a rapidly disintegrating tablets comprising Rimegepant or a pharmaceutically acceptable salt thereof and one or more pharmaceutically acceptable excipients, wherein said tablet is prepared by wet granulation method, dry granulation method or direct-compression method.

### DETAILED DESCRIPTION OF THE INVENTION

The present invention provides a rapidly disintegrating tablets composition comprising Rimegepant or a pharmaceutically acceptable salt thereof and one or more pharmaceutically acceptable excipients, processes for manufacture thereof and methods of use in the treatment or prophylaxis of disease wherein said tablet is not a freeze-dried product.

The term “rapidly disintegrating tablets” can include one or more composition(s) provided in an acceptable form for oral administration, wherein said composition which upon administration or when placed inside the oral cavity of the subject or patient, disintegrates or disperses or dissolves within about 60 seconds or less, or within about 30 seconds or less.

The term “Rimegepant” as used in the context of the present specification includes “Rimegepant” per se or its pharmaceutically acceptable salts, esters, derivatives, solvates, hydrates, enantiomers, isomers, polymorphs, prodrugs thereof. The term “pharmaceutically acceptable salt” refers to salts derived from a variety of organic and inorganic counter ions including sulfate, hemisulfate, fumarate, maleate, phosphate, tosylate, L-tartrate, citrate, acetate and oxalate. According to the present invention, Rimegepant sulfate is being preferred.

According to the present invention, Rimegepant is present in an amount from 10 to about 80% by weight based on the total weight of the composition, preferably Rimegepant is present in the form of sulfate salt in crystalline or amorphous form, preferably 15 to 50% by weight based on the total weight of the composition.

The term “pharmaceutically acceptable excipient”, means a pharmacologically inactive component such as a diluent or filler, binder, disintegrant, glidant, lubricant, taste-masking agent, or the like. The excipients that are useful in preparing a pharmaceutical composition are generally safe, non-toxic and are acceptable for veterinary as well as human pharmaceutical use.

The term “freeze dried” also known as lyophilization or cryodesiccation, is a low temperature dehydration process that involves freezing the product, lowering the pressure and transformation from ice to vapor without passing through the liquid phase.

The terms such as “about” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value. The term “about” is used to provide flexibility to a numerical range endpoint by providing that a given value may be “a little above” or “a little below” the endpoint. As used herein, the term “about” means a slight variation of the value specified, within 10 percent of the value specified. In one aspect, the present invention provides a rapidly disintegrating tablets composition comprising Rimegepant or a pharmaceutically acceptable salt thereof comprising:
(a) an intragranular portion comprising therapeutically effective amount of Rimegepant or a pharmaceutically acceptable salt thereof and at least one diluent, at least one disintegrant and one or more excipients and
(b) an extragranular portion comprising at least one disintegrant;
wherein said tablet is not freeze-dried product.

In another aspect, the present invention provides a rapidly disintegrating tablets comprising Rimegepant or a pharmaceutically acceptable salt thereof and one or more pharmaceutically acceptable excipients, wherein said tablet is prepared by wet granulation method, dry granulation method or direct-compression method.

In one aspect, the present invention provides a rapidly disintegrating tablets composition comprising Rimegepant or a pharmaceutically acceptable salt thereof comprising:
(a) an intragranular portion comprising therapeutically effective amount of Rimegepant or a pharmaceutically acceptable salt thereof and at least one diluent, at least one disintegrant and one or more excipients and
(b) an extragranular portion comprising at least one disintegrant;
wherein the ratio of diluent to disintegrant is 1:0.5 to about 1:10;
wherein said tablet is not freeze-dried product.

In one aspect the ratio of diluent to disintegrant is 1:0.5 to about 1:10, preferably 1:0.5 to about 1:8 more preferably 1:0.5 to about 1:5.

In one aspect the ratio of drug to disintegrant is 1:0.5 to about 1:10, preferably 1:0.5 to about 1:8 more preferably 1:0.5 to about 1:5.

In another embodiment, the present invention provides a rapidly disintegrating tablets composition comprising Rimegepant or a pharmaceutically acceptable salt thereof comprising:
(a) an intragranular portion comprising about 15 to 50% w/w of Rimegepant or a pharmaceutically acceptable salt thereof and about 5% w/w to about 60% w/w at least one diluent, about 1% w/w to about 10% w/w disintegrant and one or more excipients;
(b) an extragranular portion comprising about 20% w/w to about 85% w/w disintegrant;
wherein said tablet is not freeze-dried product.

In another embodiment, the present invention provides a rapidly disintegrating tablets composition comprising 15 to 50% w/w of Rimegepant or a pharmaceutically acceptable salt thereof and about 20% w/w to about 85% w/w disintegrant.

In another embodiment, the present invention provides a rapidly disintegrating tablets composition comprising Rimegepant or a pharmaceutically acceptable salt thereof comprising:
(a) an intragranular portion comprising
about 15% w/w to about 50% w/w of Rimegepant or a pharmaceutically acceptable salt thereof and
about 5% w/w to about 60% w/w diluent,
about 1% w/w to about 10% w/w disintegrating agent,
about 0.5% w/w to about 10% w/w binding agent and
(b) an extragranular portion comprising
about 20% w/w to about 85% w/w disintegrating agent,
about 0.5% w/w to about 1% w/w lubricating agent,
about 1% w/w to about 5% w/w glidant and
about 0.01% w/w to about 5% w/w taste-masking agent;
wherein said tablet is not freeze-dried product.

In another embodiment, the present invention provides a rapidly disintegrating tablets composition comprising Rimegepant or a pharmaceutically acceptable salt thereof comprising:
about 15% w/w to about 50% w/w of Rimegepant or a pharmaceutically acceptable salt thereof and
about 5% w/w to about 60% w/w diluent,
about 20% w/w to about 85% w/w disintegrating agent,
about 0.5% w/w to about 10% w/w binding agent and
about 0.5% w/w to about 1% w/w lubricating agent,
about 1% w/w to about 5% w/w glidant and
about 0.01% w/w to about 5% w/w taste-masking agent;
wherein said tablet is not freeze-dried product.

The term “disintegrating agent” or “disintegrant” refers to a water swellable polymeric material that takes up water and swells rapidly in contact with water, or when administered to a patient in less than 2 ml saliva. The water-swellable polymeric material may be present in the composition disclosed herein a variety of concentrations. For example, the rapidly disintegrating oral tablets disclosed herein can comprise water-swellable polymeric material at least 20% by weight, at least 25% by weight, at least 30% by weight, at least 35% by weight, at least 40% by weight, at least 45% by weight, at least 50% by weight, at least 60% by weight, at least 70% by weight, at least 80% by weight, at least 85% by weight or a percentage between any two of the above values, based on the total weight of the composition.

Suitable water-swellable polymeric material or disintegrants include modified starches such as directly compressible starch available under the brand name Startab®, sodium starch glycolate, croscarmellose sodium, crospovidone, low substituted hydroxypropyl cellulose and mixtures thereof.

Suitable examples of binders include, but not limited to group comprising of pregelatinized starch; partially pregelatinized starch; gelatin bloom 80, fish gelatin, cellulose or a derivative thereof such as microcrystalline cellulose, methyl cellulose, ethyl cellulose, sodium carboxymethyl cellulose, hydroxyethyl cellulose, hydroxyethyl methyl cellulose, hydroxypropyl methyl cellulose and mixture thereof. Preferably the binders according to present invention is pregelatinized starch and/or gelatin bloom 80, fish gelatin.

The binder may be present in the composition disclosed herein a variety of concentrations. The binder according to present invention may be present in an amount from about 0.5% to about 10.0% by weight with respect to total weight of the pharmaceutical composition.

Suitable examples of lubricants include, but are not limited to stearic acid, zinc stearate, sodium stearyl fumarate, magnesium stearate, sucrose stearate, aluminum stearate, adipic acid, carnauba wax, glycerol ester of fatty acid, calcium stearate, glycerol tribehenate, glyceryl behenate, polyethylene glycol and mixtures thereof. Preferably the lubricant according to present invention is magnesium stearate.

The lubricant may be present in the composition disclosed herein a variety of concentrations. Lubricant is present according to the present invention in an amount from about 0.1% to about 5% w/w.

Suitable examples of glidant include, but are not limited to talc, colloidal silicon dioxide, calcium silicate, magnesium silicate, magnesium trisilicate, fumed silica, bentonite, magnesium carbonate, glyceryl monostearate, glyceryl palmitostearate, light anhydrous silicic acid, crystalline cellulose, calcium phosphate tribasic and a combination thereof. Preferably the glidant according to present invention is colloidal silicon dioxide.

The glidant may be present in the composition disclosed herein a variety of concentrations. Glidant is present according to the present invention in an amount from about 0.1% to about 5% w/w.

Suitable examples of filers or diluents used in in various pharmaceutical compositions include, but are not limited to, microcrystalline cellulose, sodium alginate, silicified microcrystalline cellulose, microfine cellulose, mannitol, maltitol, lactose, anhydrous lactose, lactose monohydrate, spray dried lactose, maltodextrin, isomalt, dicalcium phosphate, dibasic calcium phosphate, tribasic calcium phosphate, sucrose, dextrose, magnesium carbonate and mixtures thereof.

The diluent or filer may be present in the composition disclosed herein a variety of concentrations. For example, the rapidly disintegrating oral composition disclosed herein can comprise diluent or filer at is present according to the present invention in an amount from about about 5% w/w to about 60% w/w Preferably, the amount of diluent is from about 10% to about 40% w/w, more preferably, the amount of diluent is from about 20% to about 30% w/w.

Suitable examples of taste-masking agents include, but are not limited to, the essential oils or water soluble extracts of menthol, wintergreen, peppermint, sweet mint, spearmint, vanillin, cherry, butterscotch, chocolate, cinnamon, clove, lemon, orange, raspberry, rose, spice, violet, herbal, fruit, strawberry, grape, pineapple, vanilla, peppermint, peach, kiwi, papaya, mango, coconut, apple, coffee, plum, watermelon, nuts, green tea, grape fruit, banana, butter, chamomile, sugar, dextrose, lactose, mannitol, sucrose, sucralose, xylitol, malitol, aspartame, saccharin, sorbitol, sodium saccharin, sodium cyclamate, acesulfame, honey and mixtures thereof.

The taste-masking agents may be present in the composition disclosed herein a variety of concentrations taste-masking agents is present according to the present invention in an amount from about 0.01% to about 5% w/w.

In yet another embodiment, the present invention provides a rapidly disintegrating tablets composition comprising Rimegepant or a pharmaceutically acceptable salt thereof and one or more pharmaceutically acceptable excipients, wherein said tablet is prepared by wet granulation method, dry granulation method or direct-compression method.

In yet another embodiment, the present invention provides a rapidly disintegrating tablets composition comprising Rimegepant or a pharmaceutically acceptable salt thereof and one or more pharmaceutically acceptable excipients, wherein said tablet disintegrates in saliva within 60 seconds more preferably within 30 seconds.

In yet another embodiment, the present invention provides a rapidly disintegrating tablets composition comprising Rimegepant or a pharmaceutically acceptable salt thereof and one or more pharmaceutically acceptable excipients, wherein about 90% of the Rimegepant or its salt have a particle size of not more than 150 micron; preferably not more than 120 micron.

In another embodiment, the present invention provides a rapidly disintegrating tablets composition comprising Rimegepant or a pharmaceutically acceptable salt thereof wherein the hardness of tablet is about 1 kiloponds (kp) to about 10 kiloponds (kp) more preferably about 3 kiloponds (kp) to about 10 kiloponds (kp).

In another embodiment, the present invention relates to process of preparing a rapidly disintegrating tablets composition comprising Rimegepant or a pharmaceutically acceptable salt thereof and one or more pharmaceutically acceptable excipients, wherein said tablet is not freeze-dried product.

In yet another embodiment of the present invention it provides use of a rapidly disintegrating tablets composition comprising Rimegepant in the manufacture of a medicament for treating acute treatment of migraine with or without aura in adults and preventive treatment of episodic migraine in adults.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

Example 1-5: Rimegepant rapidly disintegrating tablets.

TABLE 1

| | mg | | | | |
|---|---|---|---|---|---|
| | Examples | | | | |
| Ingredients | 1 | 2 | 3 | 4 | 5 |

Intragranular

| | | | | | |
|---|---|---|---|---|---|
| Rimegepant Sulfate | 85.67 | 85.67 | 85.67 | 85.67 | 85.67 |
| Microcrystalline | 20.00 | 90.00 | 90.00 | 90.00 | 90.00 |
| cellulose | | | | | |
| Crospovidone | 12.00 | 20.00 | 20.00 | 20.00 | 20.00 |

Binder solution

| | | | | | |
|---|---|---|---|---|---|
| Pregelatinized starch | 2.00 | 3.00 | | — | — |
| Gelatin bloom 80/ | — | — | 3.90 | 8.00 | 3.90 |
| Fish Gelatin | | | | | |
| Purified water | qs | qs | | qs | qs |

Extragranular

| | | | | | |
|---|---|---|---|---|---|
| Mannitol | — | — | 78.07 | | |
| Directly compressible | 252.82 | 129.57 | — | 79.98 | 78.67 |
| starch | | | | | |
| crospovidone | 16.15 | 10.00 | 10.00 | 9.99 | 10.00 |
| Colloidal silicon | 12.00 | 6.00 | 6.00 | 6.00 | 6.00 |
| dioxide | | | | | |
| Sucralose | 0.96 | 0.96 | 0.96 | 0.96 | 0.96 |
| Mint | 0.80 | 1.20 | 0.80 | 0.80 | 1.20 |
| Lemon | 0.80 | 0.30 | 0.80 | 0.80 | 0.30 |
| Vanilla | 0.80 | 0.30 | 0.80 | 0.80 | 0.30 |
| Magnesium stearate | 4.00 | 3.00 | 3.00 | 3.00 | 3.00 |
| Total weight | 408.00 | 350.00 | 300.00 | 306.00 | 300.00 |

Examples of 1-5 were manufactured according to the process mentioned below:
a) Co-sift Rimegepant sulfate, Crospovidone XL and Microcrystalline Cellulose through appropriate sieve.
b) load the sifted material into Rapid Mixer Granulator (RMG) for 10 mins mix with impeller at slow speed.
c) heat Purified water to 45° C.-55° C. in suitable container and add gelatin/pregelatinized starch under continuous stirring and continue stirring until to get clear solution/dispersion.
d) knead the wet mass of for 30-60 seconds with impeller at slow speed and chopper off to form granules.
e) dry the wet mass at an inlet temperature of 50+5°;
f) sift the Dried granules of step e through appropriate sieve
g) Blend the dried granules with Directly compressible starch, crospovidone colloidal silicondioxide, sucralose, mint, lemon and vanilla in low shear blender for 10 minutes and then add magnesium stearate and lubricate for 5 minutes;
h) Compress the lubricated blend into tablets.

Disintegration Test: The resulting tablets of example 1-5 were subjected to disintegration test by placing one tablet in each of the 6 tubes of the basket rack and operated the apparatus using water as the immersion fluid maintained at a temperature of 37±2° C. and found all the tablets have disintegrated completely within 30 sec.

TABLE 2
Result of hardness and friability of example 1-5 are shown below:

| Parameters | 1 | 2 | 3 | 4 | 5 |
|---|---|---|---|---|---|
| Hardness | 4.0-5.7 | 3.95-5.10 | 4.8-5.4 | 4.0-5.7 | 4.6-5.3 |
| Friability | 0.45 | 0.07 | 0.15 | Nil | Nil |

TABLE 3
Disintegration and In-vitro dissolution studies of example 2

| | | Media: pH 4.5 (50 mM sodium Acetate | | | |
|---|---|---|---|---|---|
| Dissolution | | buffer), 500 ml, Paddle, 75 RPM | | | |
| Time points | | Avg(min- | Avg(min- | Avg(min- | Avg(min- |
| (min) | Limits | Max) | Max) | Max) | Max) |
| 5 | Not less than | 97(95- | 97(94- | 96(95- | 95(93- |
| | 80% (Q) of | 98) | 100) | 97) | 96) |
| 10 | the labeled | 100(98- | 100(98- | 99(98- | 98(96- |
| | amount of | 100) | 102) | 100) | 99) |
| 15 | Rimegepant | 100(98- | 100(98- | 99(98- | 98(97- |
| | is dissolved | 101) | 102) | 100) | 99) |
| 20 | in 15 minutes. | 100(98- | 100(98- | 99(99- | 98(97- |
| | | 101) | 102) | 100) | 99) |
| 30 | | 100(99- | 100(98- | 99(98- | 98(97- |
| | | 101) | 102) | 101) | 100) |
| Disintegration | NMT 30 sec | 18 sec | 16 sec | 15 sec | 18 sec |
| Time | | | | | |

TABLE 4
stability studies of example 2

| | | | | | PVC/PVDC |
|---|---|---|---|---|---|
| | | | Cold form 3 layer | | 120 GSM |
| | | | 40° C./75% RH | | |
| Pack | | | 1.5 | 3 | 6 |
| Conditions | Limits | Initial | Months | Months | Months |
| Assay | 90-110 | 100.1 | 102.2 | 101.4 | 100.3 |
| Water/ | NMT | 6.90 | 5.93 | 6.13 | 7.46 |
| KF | 9.0% | | | | |

TABLE 5
Impurity Profile Study of example 2

| Organic | | | 1.5 | 3 | 6 |
|---|---|---|---|---|---|
| Impurities | Limits | Initial | Months | Months | Months |
| AminoHydroxy | NMT | ND | 0.01 | 0.00 | 0.00 |
| compound | 0.26 | | | | |
| Rimegepant | NMT | 0.01 | 0.02 | 0.02 | 0.02 |
| N-Oxide | 0.26 | | | | |
| Any unspecified | NMT | 0.05 | 0.05 | 0.06 | 0.09 |
| degradation | 0.2 | | | | |
| product | | | | | |
| Total impurities | NMT | 0.09 | 0.10 | 0.11 | 0.14 |
| | 0.8 | | | | |

We claim:

1. A rapidly disintegrating tablet composition comprising Rimegepant or a pharmaceutically acceptable salt thereof comprising:
(a) an intragranular portion comprising therapeutically effective amount of Rimegepant or a pharmaceutically acceptable salt thereof and at least one diluent, at least one disintegrating agent and one or more excipients, and
(b) an extragranular portion comprising at least one disintegrating agent, wherein said tablet is not a freeze-dried product.

2. The rapidly disintegrating tablet composition according to claim 1, wherein the ratio of drug to disintegrating agent is 1:0.5 to about 1:5.

3. The rapidly disintegrating tablet composition according to claim 1, wherein said tablet is prepared by wet granulation method, dry granulation method or direct-compression method.

4. The rapidly disintegrating tablet composition according to claim 1, comprising:
(a) an intragranular portion comprising:
about 15% w/w to about 50% w/w of Rimegepant or a pharmaceutically acceptable salt thereof,
about 5% w/w to about 60% w/w at least one diluent, and about 1% w/w to about 10% w/w disintegrating agent, and
(b) an extragranular portion comprising
about 20% w/w to about 85% w/w disintegrating agent,
wherein said tablet is not a freeze-dried product.

5. The rapidly disintegrating tablet composition according to claim 1, wherein diluent is selected from the group comprising of microcrystalline cellulose, sodium alginate, silicified microcrystalline cellulose, microfine cellulose, mannitol, maltitol, lactose, anhydrous lactose, lactose monohydrate, spray dried lactose, maltodextrin, isomalt, dicalcium phosphate, dibasic calcium phosphate, tribasic calcium phosphate, sucrose, dextrose, magnesium carbonate and mixtures thereof.

6. The rapidly disintegrating tablet composition according to claim 1, wherein disintegrating agent is selected from the group comprising of modified starches, sodium starch glycolate, croscarmellose sodium, crospovidone, low substituted hydroxypropyl cellulose and mixtures thereof.

7. The rapidly disintegrating tablet composition according to claim 1, further comprises: one or more binding agent, one or more lubricating agent, glidant and taste-masking agent.

8. A rapidly disintegrating tablet composition, comprising:
about 15% w/w to about 50% w/w of Rimegepant or a pharmaceutically acceptable salt thereof,
about 5% w/w to about 60% w/w diluent,
about 20% w/w to about 85% w/w disintegrating agent,
about 0.5% w/w to about 10% w/w binding agent and
about 0.01% w/w to about 5% w/w taste-masking agent;
wherein said tablet is not a freeze-dried product.

9. The rapidly disintegrating tablet composition according to claim 8 wherein diluent is selected from the group comprising of microcrystalline cellulose, sodium alginate, silicified microcrystalline cellulose, microfine cellulose, mannitol, maltitol, lactose, anhydrous lactose, lactose monohydrate, spray dried lactose, maltodextrin, isomalt, dicalcium phosphate, dibasic calcium phosphate, tribasic calcium phosphate, sucrose, dextrose, magnesium carbonate and mixtures thereof.

10. The rapidly disintegrating tablet composition according to claim 8, wherein disintegrating agent is selected from the group comprising of modified starches, sodium starch glycolate, croscarmellose sodium, crospovidone, low substituted hydroxypropyl cellulose and mixtures thereof.

11. The rapidly disintegrating tablet composition according to claim 8, wherein binder is selected from group comprising of pregelatinized starch; partially pregelatinized starch; gelatin bloom 80, fish gelatin, cellulose or a derivative thereof such as microcrystalline cellulose, methyl cellulose, ethyl cellulose, sodium carboxymethyl cellulose, hydroxyethyl cellulose, hydroxyethyl methyl cellulose, hydroxypropyl methyl cellulose and mixture thereof.

12. The rapidly disintegrating tablet composition according to claim 8, wherein taste-masking agents is selected from group comprising of essential oils or water soluble extracts of menthol, wintergreen, peppermint, sweet mint, spearmint, vanillin, cherry, butterscotch, chocolate, cinnamon, clove, lemon, orange, raspberry, rose, spice, violet, herbal, fruit, strawberry, grape, pineapple, vanilla, peppermint, peach, kiwi, papaya, mango, coconut, apple, coffee, plum, watermelon, nuts, green tea, grape fruit, banana, butter, chamomile, sugar, dextrose, lactose, mannitol, sucrose, sucralose, xylitol, malitol, aspartame, saccharin, sorbitol, sodium saccharin, sodium cyclamate, acesulfame, honey and mixtures thereof.

13. The rapidly disintegrating tablet composition according to claim 1, wherein said tablet disintegrates in saliva within 60 seconds, more preferably within 30 seconds.

14. The rapidly disintegrating tablet composition according to claim 1, wherein 90% of the Rimegepant or its salt have a particle size of not more than 150 micron; preferably not more than 120 micron.

15. The rapidly disintegrating tablet composition according to claim 1, wherein the hardness of tablet is about 1 kiloponds (kp) to about 10 kiloponds (kp) more preferably about 3 kiloponds (kp) to about 10 kiloponds (kp).

16. The rapidly disintegrating tablet composition according to claim 1, wherein the composition has total impurities of not more than 0.2% when stored at 40° C./75% RH for the period of 6 months.

17. A rapidly disintegrating tablet composition comprising 15 to 50% w/w of Rimegepant or a pharmaceutically acceptable salt thereof and about 20% w/w to about 85% w/w disintegrant.

18. The rapidly disintegrating tablet composition according to claim 8, prepared by a process comprises the steps of;
a) blending rimegepant sulfate with diluent and disintegrant into rapid mixer granulator;
b) adding binder under continuous stirring to get clear solution/dispersion;
c) granulating blended mass and further drying followed by sifting the dried granules;
d) blending the dried granules with disintegrant, glidant and taste-masking agents;
and compressing the lubricated blend into tablets.

19. The rapidly disintegrating tablet composition according to claim 1, wherein the composition is used for the acute treatment of migraine with or without aura in adults and preventive treatment of episodic migraine in adults.
